(19) United States
(12) Patent Application Publication
(10) Pub. No.: US 2014/0277062 A1
(43) Pub. Date: Sep. 18, 2014

(54) MEDICAL BALLOON HAVING TAPERED OR STEPPED PROFILE

(71) Applicant: FUTUREMATRIX INTERVENTIONAL, INC., (US)

(72) Inventors: LANNY R. PEPPER, LARUE, TX (US); CHARLES J. COX, EUSTACE, TX (US)

(73) Assignee: FUTUREMATRIX INTERVENTIONAL, INC., ATHENS, TX (US)

(21) Appl. No.: 13/828,979

(22) Filed: Mar. 14, 2013

(51) Int. Cl. A61M 29/02

(52) U.S. Cl. CPC A61M 29/02
USPC 606/192

(57) ABSTRACT

A medical balloon that may be inflated and deflated includes a barrel wall having a large diameter proximal end and a small diameter distal end, the barrel wall being disposed between proximal and distal tapered cone walls, each having proximal and distal ends, and proximal and distal cylindrical neck walls extending therefrom along a longitudinal axis and wherein the barrel wall is continuously tapered inwardly toward a longitudinal axis of the balloon from the large diameter proximal end of the barrel wall to the small diameter distal end of the barrel wall.

### TECHNICAL FIELD

This disclosure relates to the field of medical balloons. In particular, it relates to medical balloons that are useful in angioplasty and other medical applications including cardiology, radiology, urology and orthopedics.

### BACKGROUND

Non-compliant medical balloons for performing angioplasty and other medical procedures are known. U.S. Pat. No. 6,746,425 to Beckham discloses a semi-compliant medical balloon and methods for manufacturing the balloon. U.S. Patent Application Publication No. US 2006/0085022 to Hayes et al. discloses a semi-compliant medical balloon having an integral woven fabric layer and methods for manufacturing the balloon. U.S. Patent Application Publication No. US 2006/0085023 to Davies, Jr. et al. discloses a medical balloon having strengthening rods and methods for manufacturing the balloon. U.S. Patent Application Publication No. US 2006/0085024 to Pepper et al. discloses a semi-compliant medical balloon having an integral non-woven fabric layer and methods for manufacturing the balloon. U.S. Pat. No. 6,746,425 and Publication Nos. US 2006/0085022, US 2006/0085023 and US 2006/0085024 are incorporated herein by reference.

In a common situation, a medical balloon, mounted on a catheter shaft, is inserted into the body via an introducer and guided through the vascular system. When the balloon reaches the desired treatment site within a body lumen, a fluid is injected through an inflation lumen in the catheter shaft to inflate the balloon. The balloon expands in an outward (i.e., radial) direction as it inflates, thereby dilating the body lumen at the treatment site. The fluid may then be withdrawn from the balloon via the inflation lumen, causing the balloon to deflate so that it can be moved to the next treatment site or withdrawn.

Placement of a medical dilation balloon may require that the balloon be forced into and/or through a body lumen of varying diameters, for example through a narrowing blood vessel or a blood vessel with an occlusion that reduces the diameter of the vessel in a particular location. For example, medical dilation balloons may be used to perform peripheral, for example, below-the knee, angioplasty to open a stenosis or occlusion of an artery, with or without stent placement. In some instances, this procedure requires that the balloon be threaded through small blood vessels along a tortuous path. Once in place, a balloon may be used to expand a body lumen having an occlusion where a portion of the balloon is expanded against the occlusion while a portion of the balloon is in a non-occluded region of the body lumen. Consequently, a number of characteristics are desired for medical dilation balloons used in these procedures.

In some instances, multiple expansions to different balloon diameters may be necessary or desirable. For example, when expanding a narrow or occluded blood vessel, it may be necessary to initially use a balloon having a relatively small diameter in order to maneuver the balloon into the desired location. The balloon is then inflated to its maximum diameter, deflated and removed. If the desired amount of expansion is not achieved, a balloon with a larger inflated diameter is then introduced, maneuvered into position and inflated. This process may be repeated multiple times to achieve the desired amount of expansion. However, introducing, placing and expanding multiple balloons having successively larger diameters is time consuming and adds to the potential for complications during the procedure.

### SUMMARY

A medical balloon that may be inflated and deflated includes a barrel wall having a large diameter proximal end and a small diameter distal end, the barrel wall being disposed between proximal and distal tapered cone walls, each having proximal and distal ends. Proximal and distal cylindrical neck walls extend proximally and distally from the proximal end of the proximal cone wall and from the distal end of the distal cone wall, respectively. The barrel wall of the balloon is continuously tapered inwardly toward a longitudinal axis of the balloon from the large diameter proximal end of the barrel wall to the small diameter distal end of the barrel wall. The diameter of the proximal end of the barrel wall is from 3 mm to about 6 mm and the diameter of the distal end of the barrel wall is from 1 mm to about 3 mm. In different embodiments, the ratio of the diameter of the proximal end of the barrel wall to the diameter of the distal end of the barrel wall is from 3:1 to about 5:2. In one variation, the length of the barrel wall may range from 4 cm to about 10 cm; in another, from 1 cm to about 30 cm. In one aspect, the medical balloon may be formed from one of Nylon 6, Nylon 11 and Nylon 12, or a polyether block amide; in another, the medical balloon is formed from a crystalline polyethylene terephthalate.

In an alternate embodiment, the medical balloon has a barrel wall with a large diameter proximal end and a small diameter distal end. The barrel wall is disposed between proximal and distal tapered cone walls, each having proximal and distal ends, with proximal and distal cylindrical neck walls extending therefrom along a longitudinal axis of the balloon. The barrel wall includes a proximal section having a first diameter and at least one reduced diameter section extending distally from the proximal section of the barrel wall, with the proximal section and the reduced diameter section each having a constant diameter section along their respective lengths. In one aspect, the barrel wall includes a plurality of stepped diameter sections including a proximal stepped diameter section having a first diameter and a plurality of successive, distally extending stepped diameter sections. Each of the successive, distally extending stepped diameter sections has a diameter smaller than the diameter of the preceding proximal stepped diameter section. The diameter of each of the stepped diameter sections is constant along the length of the section. In one embodiment, the difference in diameter between each of the plurality of successive, distally extending stepped diameter sections and the preceding proximal stepped diameter section is at least 1 mm. The diameter of the proximal stepped diameter section may be in the range of from 3 mm to about 6 mm while the diameter of the distally endmost stepped diameter section is from 1 mm to about 3 mm. In one embodiment, the ratio of the diameter of the proximal stepped diameter section to the diameter of the distally endmost stepped diameter section is from about 3:1 to about 5:2. In another aspect, a truncated conical section or transition cone extends between at least two of the plurality of successive, distally extending stepped diameter sections of the barrel section of balloon.

### BRIEF DESCRIPTION OF THE DRAWINGS

For a more complete understanding, reference is now made to the following description taken in conjunction with the accompanying Drawings in which:

FIG. 1 is a side view of an inflated tapered diameter balloon according to the disclosure;

FIG. 2 is a side view of an inflated stepped diameter balloon according to the disclosure; and

FIG. 3 is a side view of an alternate embodiment of the stepped diameter balloon of FIG. 2.

### DETAILED DESCRIPTION

Referring now to the drawings, wherein like reference numbers are used herein to designate like elements throughout, the various views and embodiments of a medical balloon having tapered or stepped profile are illustrated and described, and other possible embodiments are described. The figures are not necessarily drawn to scale, and in some instances the drawings have been exaggerated and/or simplified in places for illustrative purposes only. One of ordinary skill in the art will appreciate the many possible applications and variations based on the following examples of possible embodiments.

Referring now to FIG. 1, there is illustrated a medical balloon 100 in accordance with one aspect, shown in its fully inflated state. Balloon 100 includes a barrel portion 102 disposed between tapered cone portions 104 and cylindrical neck portions 106 extending from the cone portion along a longitudinal axis 108. The outer surface 110 of each cone portion 104 forms an angle α (the “cone angle”) with respect to longitudinal axis 108 (or with respect to a longitudinal extension of the wall of the barrel portion 102 parallel to the longitudinal axis). Different embodiments of the balloon 100 may have a cone angle α in the range of 12 degrees to 30 degrees. A higher cone angle α results in shorter overall length of the balloon for a given barrel length. In some embodiments, balloon 100 may have a cone angle α in the range of 12 degrees to 22 degrees; in others from 18 degrees to 22 degrees. In some embodiments, the cone angle α is about 20 degrees. A guidewire lumen 120 may extend through the interior of the balloon between the neck portions 106 and may include inflation/deflation ports (not shown).

As illustrated, barrel portion 102 tapers continuously inward (toward longitudinal axis 108) from a large diameter proximal end 116 having a diameter D1 to a small diameter distal end 118 having a diameter D2. In different embodiments, the ratio of the diameter of the large diameter proximal end 116 to the small diameter distal end 118 (D1:D2) may range from about 3:1 to about 5:2. The diameter D1 of the large diameter proximal end 116 may typically range from 3 mm to about 6 mm. The diameter D2 of the small diameter distal end 118 may typically vary from 1 mm to about 3 mm. The diameters referred to herein are outside diameters unless otherwise noted. Thus, in different variations, the diameter ratio D1:D2 may be 3 mm:1 mm, 5 mm:2 mm, 6 mm:3 mm or similar ratios. The length L of barrel portion 102 of balloon 100 (the working length of balloon 100) is typically from about 4 cm to about 10 cm; however, depending upon the particular application, length L may range from 1 cm to 30 cm.

As illustrated, the taper of barrel portion 102 is uniform along the length of the barrel portion between tapered cone portions 104. Thus, a balloon 100 with a barrel portion 102 having a D1:D2 ratio of 5 mm:3 mm would have a diameter D3 of 4 mm midway along its length.

Balloon 100 provides a number of advantages over prior art balloons having a fixed diameter barrel section. The relatively narrow distal end of tapered balloon 100 provides for better tracking, i.e., navigation through body lumens, than a comparable fixed diameter balloon having the same overall average diameter for at least two reasons. First, the relatively narrow distal end of tapered balloon 100 is easier to guide into a narrow body lumen than the distal end of a comparable fixed diameter balloon having the same average diameter, which will necessarily have a larger diameter distal end. Second, the relatively narrow distal end of tapered balloon 100 will typically be more flexible than the distal end of a comparable fixed diameter balloon having the same average diameter. The more flexible end of tapered balloon 100 will allow easier navigation when the direction of the body lumen changes, for example when a vessel transitions to a Y juncture.

Balloon 100 also permits multiple expansions of a narrow or occluded lumen to different diameters. For example, a tapered balloon 100 having a 1 mm barrel diameter at the distal end and a 3 mm diameter at the proximal end may be guided into an occluded section of a body lumen up to the midpoint of the barrel. The tapered balloon 100 may then be inflated to its maximum diameter which, at the midpoint of barrel 102 of the balloon is 1.5 mm. If further expansion of the lumen is desired, balloon 100 may be partially deflated, if necessary, and guided further into the occluded section of lumen, for example up to the proximal end of barrel portion 102. Balloon 100 may then be re-inflated to its maximum diameter, which adjacent the proximal end of barrel 102 is 3 mm. Thus, a tapered balloon 100 may permit the occluded section of the body lumen to be expanded with a single balloon to a degree that might otherwise have required changing from a small fixed diameter balloon to a larger fixed diameter.

Barrel portion 102 of balloon 100 may include one or more radio opaque markers 122 or materials positioned along the length of the balloon to aid in positioning balloon 100 in a body lumen. In some embodiments, the radio opaque markers 122 may be incorporated into and/or affixed to the guidewire lumen 120. In other embodiments, the radio opaque markers 122 may be films or materials incorporated into and/or placed on the balloon envelope. Radio opaque markers 122 or materials incorporated in barrel portion 102 may enable the balloon to be positioned at a location wherein a selected diameter portion of the balloon may be used to expand a body lumen. For example, if the diameter of barrel section 102 tapers from 5 mm at the proximal end 118 of the barrel section to 2 mm at the distal end of the barrel section, the balloon may be positioned such that a portion of the balloon having an inflated diameter of 3 mm is located at a selected location in the body lumen where expansion to 3 mm is desired.

FIG. 2 is a side view of an inflated stepped diameter balloon 200 according to the disclosure. Balloon 200 includes a barrel portion 202 disposed between tapered cone portions 204 and cylindrical neck portions 206 extending from the cone portion along a longitudinal axis 208. As in the case of balloon 100, the outer surface 210 of each cone portion 204 of balloon 200 forms an angle α (the “cone angle”) with respect to the longitudinal axis 208. Different embodiments of the balloon 200 may have a cone angle α in the range of 12 degrees to 30 degrees. As previously noted, a greater cone angle α provides for a shorter overall length of the balloon with a given barrel length. In some embodiments, balloon 200 may have a cone angle α in the range of 12 degrees to 22 degrees, in others from 18 degrees to 22 degrees. In some embodiments, the cone angle α is about 20 degrees.

Barrel portion 202 of balloon 200 includes a series of stepped diameter sections 202a, 202b and 202c, each having a constant diameter over the length of the section. Although, barrel portion 202 of balloon 200 is illustrated with three stepped diameter sections 202a, 202b and 202c, barrel portion 202 may include a greater or lesser number, for example 2, 4, 5 or other number of stepped diameter sections. The diameter d1 of proximal stepped diameter section 202a may range from 3 mm to about 6 mm while the diameter d3 of the distally endmost stepped diameter section may range from 1 mm to about 3 mm. In different variations, the ratio of the diameter d1 of proximal stepped diameter section 202a to the diameter d3 of the distally endmost stepped diameter section 202c is in the range of from about 3:1 to about 5:2.

As illustrated, the diameter of each successive section 202b and 202c extending distally from proximal section 202a decreases incrementally from the diameter of the preceding proximal stepped diameter section. For example, the diameters d1, d2 and d3 of stepped diameter sections 202a, 202b and 202c of barrel portion 202 may be 3 mm, 2 mm and 1 mm, respectively. In a different variation, the diameters d1, d2 and d3 of sections 202a, 202b and 202c may be 5 mm, 2 mm and 1 mm, respectively. Although the difference in diameters between sections 202a, 202b and 202c may be greater or smaller than 1 mm, in most applications, the difference will be at least 1 mm. Thus, the ratio of the diameters d1, d2 and d3 of sections 202a, 202b and 202c may be 3:2:1, 5:2:1 or 6:3:2.

In one embodiment, a radio opaque marker or material 122 may be placed on the guidewire lumen 120 or incorporated in one or more of stepped diameter sections 202a, 202b and 202c to aid in positioning balloon 200 and/or one of the stepped diameter sections at a selected location in a body lumen. Stepped diameter sections 202a, 202b and 202c also provide discreet sites along the length of balloon 200 that may be located in a body lumen at a selected location for expansion to the inflated diameter of a selected one of the stepped diameter portions.

The incremental change in diameters d1, d2 and d3 between sections 202a, 202b and 202c may be the same or different. For example, the diameters d1, d2 and d3 of sections 202a, 202b and 202c may be 5 mm, 2 mm and 1 mm, respectively. The lengths l1, l2 and l3 of each section 202a, 202b and 202c may be the same or different, depending upon the particular application. For example, in one variation, the lengths l1, l2 and l3 of sections 202a, 202b and 202c may be 2 cm, 2 cm and 2 cm, respectively. Alternatively, in another variation, the lengths l1, l2 and l3 of sections 202a, 202b and 202c may be 3 cm, 2 cm and 1 cm, respectively.

Balloons 100 and 200 may be compliant, semi-compliant or non-compliant. Balloon compliance is a term used to describe the change in a balloon's diameter as a function of pressure. Non-compliant medical dilation balloons expand very little, if at all, when pressurized from a nominal diameter to a rated burst pressure. Generally, non-compliant balloons are formed from relatively inelastic materials such as oriented highly crystalline polyethylene terephthalate (PET) films. Such PET films provide high tensile strength, and may be used to form balloons with thin walls having high burst pressures. High pressure non-compliant balloons may have rated burst pressures of up to 20 atmospheres or higher.

Semi-compliant medical balloons exhibit moderate expansion when pressurized from an operating pressure (e.g., the pressure at which the balloon reaches its nominal diameter) to a rated burst pressure. Semi-compliant medical balloons may, according to one aspect, be formed from polyamides such as Nylon 6, Nylon 11 and Nylon 12, a polyether block amide (PEBA) such as PEBAX® brand PEBA. Semi-compliant balloons, while not achieving the rated burst pressure of a non-compliant balloon, may be less stiff than non-compliant balloons, and may provide better puncture resistance than non-compliant balloons. Low pressure compliant medical balloons are typically formed from elastomers such as latex, polyurethane and other thermoplastic elastomers. Low pressure compliant medical balloons may expand by 100% or greater upon inflation.

FIG. 3 is a side view of an alternate embodiment of the stepped diameter balloon of FIG. 2. As illustrated, barrel portion 202 of balloon 200 includes a truncated transition cone portion or transition cone 220a between stepped diameter sections 202a and 202b and transition cone 220b between stepped diameter sections 202b and 202c. The length and cone angle of transition cones 220a and 220b may be the same or different, depending upon the difference in the diameters d1, d2 and d3 of stepped diameter sections 202a, 202b and 202c. Other factors such as the particular application and the material of barrel portion 202 may affect the length and cone angle of transition cones 220a and 220b.

As illustrated, barrel portion 202 includes three stepped diameter sections 202a, 202b and 202c with transition cones 220a and 220b extending therebetween. In different embodiments, barrel portion 202 may include a greater or lesser number of stepped diameter sections, for example 2, 4, 5 or more with transition cones extending between all or some of the stepped diameter portions. Transition cones 220a and 220b may facilitate manufacture of balloon 200. Transition cones 220a and 220b may also provide balloon 200 greater flexibility and improved tracking. It is also contemplated that the use of a radio opaque material or marker 122 at transition cones 220a and 220b may facilitate locating balloon 200 and/or a selected one of stepped diameter sections 202a, 202b and 202c at a desired location in a body lumen.

It will be appreciated by those skilled in the art that the disclosure provides a medical balloon having tapered or stepped profile. It should be understood that the drawings and detailed description herein are to be regarded in an illustrative rather than a restrictive manner, and are not intended to be limiting to the particular forms and examples disclosed. On the contrary, included are any further modifications, changes, rearrangements, substitutions, alternatives, design choices, and embodiments apparent to those of ordinary skill in the art, without departing from the spirit and scope hereof, as defined by the following claims. Thus, it is intended that the following claims be interpreted to embrace all such further modifications, changes, rearrangements, substitutions, alternatives, design choices, and embodiments.

What is claimed is:

1. A medical balloon that may be inflated and deflated, comprising:
a barrel wall having a large diameter proximal end and a small diameter distal end, the barrel wall being disposed between proximal and distal tapered cone walls, each having proximal and distal ends, and proximal and distal cylindrical neck walls extending therefrom along a longitudinal axis; and
wherein the barrel wall is continuously tapered inwardly toward a longitudinal axis of the balloon from the large diameter proximal end of the barrel wall to the small diameter distal end of the barrel wall.

2. The medical balloon of claim 1, wherein the diameter of the proximal end of the barrel wall is from about 3 mm to about 6 mm.

3. The medical balloon of claim 1, wherein the ratio of the diameter of the distal end of the barrel wall is from about 1 mm to about 3 mm.

4. The medical balloon of claim 1, wherein the ratio of diameter of the proximal end of the barrel wall to the diameter of the distal end of the barrel wall is from about 3:1 to about 5:2.

5. The medical balloon of claim 1, wherein the length of the barrel wall is from about 4 cm to about 10 cm.

6. The medical balloon of claim 1 wherein the length of the barrel wall is from about 1 cm to about 30 cm.

7. The medical balloon of claim 1, wherein the medical balloon is formed from one of Nylon 6, Nylon 11 and Nylon 12, or a polyether block amide.

8. The medical balloon of claim 1, wherein the balloon is formed from a crystalline polyethylene terephthalate.

9. A medical balloon that may be inflated and deflated, comprising:
a barrel wall having a large diameter proximal end and a small diameter distal end, the barrel wall being disposed between proximal and distal tapered cone walls, each having proximal and distal ends, and proximal and distal cylindrical neck walls extending therefrom along a longitudinal axis of the balloon; and
wherein the barrel wall comprises a proximal section having a first diameter and at least one reduced diameter section extending distally from the proximal section of the barrel wall, the proximal section and the reduced diameter section each having a constant diameter section along its length.

10. The medical balloon of claim 9 further comprising a barrel wall including a plurality of stepped diameter sections including a proximal stepped diameter section having a first diameter and a plurality of successive, distally extending stepped diameter sections, each of the successive, distally extending stepped diameter sections each having a diameter smaller than the diameter of the preceding proximal stepped diameter section, and wherein the diameter of each of the stepped diameter sections is constant along the length of the section.

11. The medical balloon of claim 10, wherein the difference in diameter between each of the plurality of successive, distally extending stepped diameter sections and the preceding proximal stepped diameter section is at least 1 mm.

12. The medical balloon of claim 10, wherein the diameter of the proximal stepped diameter section is from about 3 mm to about 6 mm.

13. The medical balloon of claim 10, wherein the diameter of the distally endmost stepped diameter section is from about 1 mm to about 3 mm.

14. The medical balloon of claim 10, wherein the ratio of the diameter of the proximal stepped diameter section to the diameter of the distally endmost stepped diameter section is from about 3:1 to about 5:2.

15. The medical balloon of claim 9, wherein the length of the barrel wall is from about 4 cm to about 10 cm.

16. The medical balloon of claim 9, wherein the length of the barrel wall is from about 1 cm to about 30 cm.

17. The medical balloon of claim 9, wherein the medical balloon is formed from one of Nylon 6, Nylon 11 and Nylon 12, or a polyether block amide.

18. The medical balloon of claim 9, wherein the balloon is formed from a crystalline polyethylene terephthalate.

19. The medical balloon of claim 9, further comprising a transition cone extending between at least two of the plurality of successive, distally extending stepped diameter sections of the barrel section of balloon.
